(19) United States
(12) Patent Application Publication
(10) Pub. No.: US 2019/0255284 A1
(43) Pub. Date: Aug. 22, 2019

(54) Fluid Delivery Systems and Methods

(71) Applicant: ALCYONE LIFESCIENCES, INC., Lowell, MA (US)

(72) Inventors: Jonathan Freund, Woburn, MA (US); PJ Anand, Lowell, MA (US); Deep Arjun Singh, Cambridge, MA (US); Gregory Eberl, Acton, MA (US)

(21) Appl. No.: 16/281,745

(22) Filed: Feb. 21, 2019

Related U.S. Application Data
(60) Provisional application No. 62/633,103, filed on Feb. 21, 2018.

(51) Int. Cl. A61M 25/00; A61J 15/00

(52) U.S. Cl. CPC A61M 25/0023; A61J 15/003; A61M 2025/091; A61M 2210/04; A61M 25/0097

(57) ABSTRACT

Catheters, catheter ports, connectors, and related methods are disclosed herein, e.g., for drug delivery to a subject. The catheters and catheter ports can include various features to facilitate dosing protocols that require multiple injections, and/or for reducing or eliminating damage that may occur to the catheter, port, or patient tissue as a result of multiple injections.

### CROSS-REFERENCE TO RELATED APPLICATION

This application claims the benefit of U.S. Provisional Application No. 62/633,103, filed Feb. 21, 2018, which is hereby incorporated by reference herein in its entirety.

### FIELD

Catheters, catheter ports, and related methods are disclosed herein, e.g., for delivering a drug to a subject, for example via intrathecal delivery into the cerebrospinal fluid (CSF) or subarachnoid space of the subject's brain or spine.

### BACKGROUND

There are many instances in which it may be desirable to deliver a drug to a patient. The term “drug” as used herein refers to any functional agent that can be delivered to a human or animal subject, including hormones, stem cells, gene therapies, chemicals, compounds, small and large molecules, dyes, antibodies, viruses, therapeutic agents, etc.

Delivery of the drug can be done in a systemic manner, or can be targeted to a particular location or a particular distribution pattern. The dosing protocol of certain drugs, including complex therapeutics for genetic disorders, may require multiple injections over a short or long period of time to various areas of the subject, such as the intrathecal space. Use of existing delivery techniques may result in damage to the skin or other tissue of the subject, or to implanted components of the delivery system.

There is a continual need for improved drug delivery systems and methods.

### SUMMARY

Catheters, catheter ports, connectors, and related methods are disclosed herein, e.g., for drug delivery to a subject. The catheters and catheter ports can include various features to facilitate dosing protocols that require multiple injections, and/or for reducing or eliminating damage that may occur to the catheter, port, or patient tissue as a result of multiple injections.

In accordance with a first aspect, a fluid delivery system is disclosed that includes an implantable port having a housing with one or more fluid openings therein and a connector that is configured to be selectively mated to the port over a skin surface of a patient. The connector has one or more openings that are configured to align with the one or more fluid openings of the port.

According to some forms, the implantable port can include a spool that is rotatably mounted in the housing, where the spool is adapted to have portions of a catheter wound therearound. In some versions, the spool can include a cylindrical body having a helical groove formed in an outer surface thereof and/or the spool can be mounted to a shaft having a longitudinal slot with a key slidably disposed therein. In some versions, the key can include an opening extending therethough and the catheter can be connected to the key and in fluid communication with the opening; the key can be configured to shift along the longitudinal slot of the shaft as the spool rotates to unwind a catheter would therearound; and/or the housing can include a groove and the shaft can include a radial flange disposed within the groove to retain the shaft to the housing. In further forms, the system can include a catheter that extends from the port and is in fluid communication with the one or more fluid openings. These forms can include one or more of the following aspects: the catheter can include a distal-facing fluid opening and a plurality of side-facing fluid openings, where the side-facing fluid openings are arranged in a helical pattern; the catheter can include a longitudinal line marker that is configured to indicate a twist of the catheter; the catheter can include markers that indicate proximal and distal ends of a region of the catheter in which side-facing fluid ports are formed; the catheter can include a plurality of markers disposed along length increments; or at least one of the catheter or the port can be designed for therapy specific applications and treatments to a disorder selected from the group consisting of Huntington's disease, Spinal Muscular Atrophy (SMA), survival motor neuron (SMN) deficiency, amyotrophic lateral sclerosis (ALS), Angelman's Syndrome, Dravet Syndrome, Alzheimer's disease, progressive supranuclear palsy (PSP), frontotemporal dementia (FTD), Parkinson's Disease, central nervous system (CNS) lymphoma, Leptomeningeal Cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN).

According to some forms, the system can include one or more of the following aspects: the one or more fluid openings of the housing can each include a respective septum; the one or more fluid openings of the housing can be in fluid communication with one another via an inner lumen of the housing; the housing and the connector can include counterpart alignment features, where, in some versions, the alignment features can be at least one of magnetic, tactile, and shape alignment; the housing can include a top surface and a side surface and the connector can include a top wall having a shape complementary to the top surface of the housing and a skirt depending downwardly from the top wall, such that with the connector selectively mated to the port over a skin surface of a patient, the skirt extends along portions of the side surface of the housing, where, in further forms, the housing can include one or more ribs protruding outwardly from the side surface thereof and the skirt of the connector can include one or more grooves each adapted to at least partially receive one of the one or more ribs therein with the connector mated to the port over a skin surface of a patient; the housing can include a male barbed catheter fitting adapted to receive a catheter thereover to fluidly couple the catheter to the housing; or the connector can include a housing have a shape complementary to a shape of the housing of the implantable port.

According to some forms, the system can include one or more dosages of a nucleic acid, a protein therapeutic, a cell therapy, a small molecule therapeutic, or a combination thereof. In one example, the system can include a nucleic acid selected from the group consisting of an antisense oligonucleotide, a ribozyme, an miRNA, an siRNA, and and shRNA, or a nucleic acid encoding a clustered regularly interspaced short palindromic repeats (CRISPR) associated protein (Cas) system, or a combination thereof. In some versions, the nucleic acid can be an antisense oligonucleotide comprising a 2′-O-2-methoxyethyl (“2′-MOE”) group; the antisense oligonucleotide can be nusinersen; or the system can include an antisense nucleic acid that targets HTT. In another example, the system can include one or more dosages of a viral vector encoding a therapeutic protein, where, in a further form, the viral vector can be an adeno-associated viral vector or an adenoviral vector. In another example, the nucleic acid, protein therapeutic, cell therapy, small molecule therapeutic, or combination thereof can treat a disorder selected from the group consisting of Huntington's disease, Spinal Muscular Atrophy (SMA), survival motor neuron (SMN) deficiency, amyotrophic lateral sclerosis (ALS), Angelman's Syndrome, Dravet Syndrome, Alzheimer's disease, progressive supranuclear palsy (PSP), frontotemporal dementia (FTD), Parkinson's Disease, central nervous system (CNS) lymphoma, Leptomeningeal Cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN).

In accordance with a second aspect, a fluid delivery system is disclosed that includes an implantable port having a housing with one or more fluid openings therein and a catheter that extends from the port and is in fluid communication with the one or more fluid openings. The catheter includes a distal-facing fluid opening and a plurality of side-facing fluid openings, the side-facing fluid openings being arranged in a helical pattern.

According to some forms, the system can include one or more of the following aspects: the catheter can include a longitudinal line marker configured to indicate a twist of the catheter; the catheter can include markers that indicate proximal and distal ends of a region of the catheter in which the side-facing fluid openings are formed; the catheter can include a plurality of markers disposed along length increments; the housing can include a male barbed catheter fitting adapted to receive a catheter thereover to fluidly couple the catheter to the housing; the one or more fluid openings of the housing can each include a respective septum; the implantable port can include a spool rotatably mounted in the housing, where the spool adapted to have portions of the catheter wound therearound and, in a further form, can include a cylindrical body having a helical groove formed in an outer surface thereof and/or be mounted to a shaft having a longitudinal slot with a key slidably disposed therein; or at least one of the catheter or the port can be designed for therapy specific applications and treatments to a disorder selected from the group consisting of Huntington's disease, Spinal Muscular Atrophy (SMA), survival motor neuron (SMN) deficiency, amyotrophic lateral sclerosis (ALS), Angelman's Syndrome, Dravet Syndrome, Alzheimer's disease, progressive supranuclear palsy (PSP), frontotemporal dementia (FTD), Parkinson's Disease, central nervous system (CNS) lymphoma, Leptomeningeal Cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN).

In accordance with a third aspect, a fluid delivery system is disclosed that includes an implantable catheter, means for establishing fluid communication between the implantable catheter and a needle, and means for aligning the needle with the means for establishing fluid communication.

### BRIEF DESCRIPTION OF THE DRAWINGS

FIG. 1 is a schematic diagram of a delivery system at least partially implanted in a patient according to various embodiments of the disclosure;

FIG. 2A is a sectional view of a catheter suitable for use in the system of FIG. 1;

FIG. 2B is a sectional view of a catheter distal end suitable for use in the system of FIG. 1;

FIG. 2C is a sectional view of a catheter having steering features suitable for use in the system of FIG. 1;

FIG. 2D is a sectional view of an intermediate portion of a catheter suitable for use in the system of FIG. 1;

FIG. 2E is a sectional view of an intermediate portion of a catheter suitable for use in the system of FIG. 1;

FIG. 3A is a top perspective view of a first example port suitable for use in the system of FIG. 1;

FIG. 3B is a bottom perspective view of the port of FIG. 3A;

FIG. 3C is a top plan view of the port of FIG. 3A;

FIG. 3D is a bottom plan view of the port of FIG. 3A;

FIG. 3E is a cross-sectional view of the port of FIG. 3A;

FIG. 3F is a side elevational view of the port of FIG. 3A;

FIG. 3G is a cross-sectional view of the port of FIG. 3A;

FIG. 3H is a schematic view of the port of FIG. 3A and a connector configured to mate with the port;

FIG. 3I is a cross-sectional view of the port of FIG. 3A;

FIG. 3J is a perspective view of a second example port suitable for use in the system of FIG. 1;

FIG. 4A is a top perspective view of a connector suitable for use in the system of FIG. 1;

FIG. 4B is a bottom perspective view of the connector of FIG. 4A;

FIG. 4C is a side elevational view of the connector of FIG. 4A;

FIG. 4D is a top plan view of the connector of FIG. 4A;

FIG. 5A is a top perspective view of another example port suitable for use in the system of FIG. 1;

FIG. 5B is a bottom perspective view of the port of FIG. 5A;

FIG. 5C is an exploded view of the port of FIG. 5A;

FIG. 5D is a diagrammatic view of the port of FIG. 5A;

FIG. 5E is a side elevational view of a spool of the port of FIG. 5A;

FIG. 5F is a perspective view of the spool of FIG. 5E;

FIG. 6A is a schematic view of another example port and connector suitable for use in the system of FIG. 1;

FIG. 6B is a top perspective view of the connector of FIG. 6A;

FIG. 6C is a bottom perspective view of the connector of FIG. 6A;

FIG. 6D is a top perspective view of the port of FIG. 6A;

FIG. 6E is an exploded, cross-sectional view of the port of FIG. 6A;

FIG. 6F is a diagrammatic view of the port of FIG. 6A;

FIG. 6G is a side elevational view of the port of FIG. 6A;

FIG. 6H is a cross-sectional view of the port of FIG. 6A;

FIG. 6I is a perspective view of a shaft of the port of FIG. 6A;

FIG. 6J is a perspective view of a shaft and spool of the port of FIG. 6A;

FIG. 7A is a schematic view of another example port and connector configured to mate with the port; and

FIG. 7B is a bottom perspective view of the connector of FIG. 7A.

### DETAILED DESCRIPTION

Catheters, catheter ports, and related methods are disclosed herein, e.g., for drug delivery to a subject. The catheters and catheter ports can include various features to facilitate dosing protocols that require multiple injections, and/or for reducing or eliminating damage that may occur to the catheter, port, or patient tissue as a result of multiple injections.

Certain exemplary embodiments will now be described to provide an overall understanding of the principles of the structure, function, manufacture, and use of the methods, systems, and devices disclosed herein. One or more examples of these embodiments are illustrated in the accompanying drawings. Those skilled in the art will understand that the methods, systems, and devices specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure.

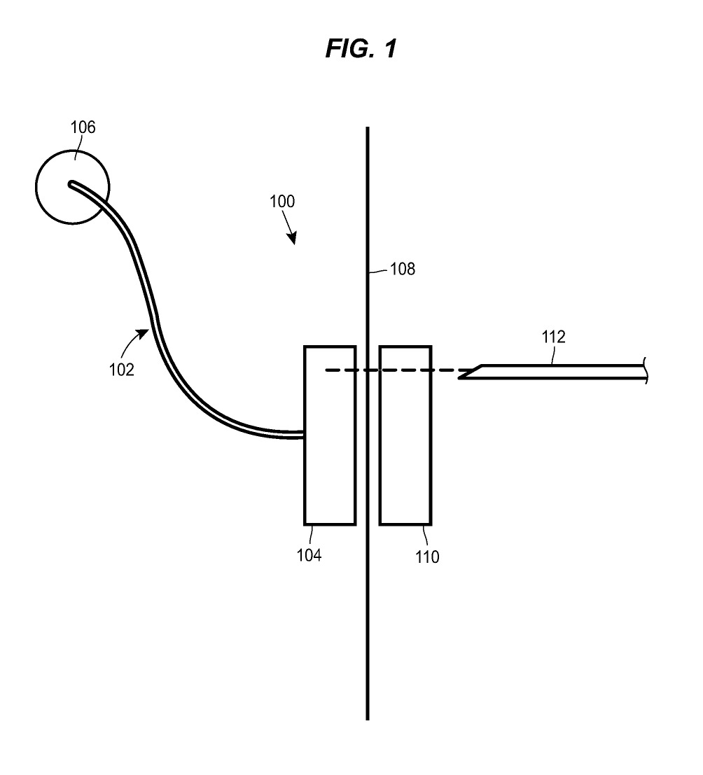

FIG. 1 illustrates an exemplary delivery system 100. As shown, the system 100 can include an implantable catheter 102 coupled to an implantable port 104. The catheter 102 can be implanted in a patient to position at least a portion of the catheter 102 in or near a target site 106. The target site 106 can be any of a variety of locations within a patient, such as the brain, spine, intrathecal space, subarachnoid space, subdural space, etc. The port 104 can be implanted in the patient, for example just beneath the patient's skin 108. The system 100 can include a connector 110 configured to mate with the implanted port 104 over the patient's skin 108. The connector 110 can serve as an alignment guide for delivering material (e.g., a drug or chaser) to the implanted port 104 or for removing material (e.g., CSF or drug) from the implanted port. For example, the connector 110 can include one or more openings configured to receive a needle 112 therethrough. When mated to the port 104 over the patient's skin 108, the one or more openings of the connector 110 can be aligned with one or more fluid ports or injection sites of the port. As discussed below, the connector 110 can include various features for facilitating alignment with the implanted port 104. In use, the connector 110 can be positioned over the patient's skin 108, above the implanted port 104, to mate the connector to the port. A needle or other delivery device 112 can then be inserted through an opening of the connector 110, through the patient's skin 108, and into the implanted port 104 to establish fluid communication between the catheter 102 and the needle. Fluid can then be delivered to the target site 106 via the needle 112 and the catheter 102, or can be removed from the target site via the needle and the catheter.

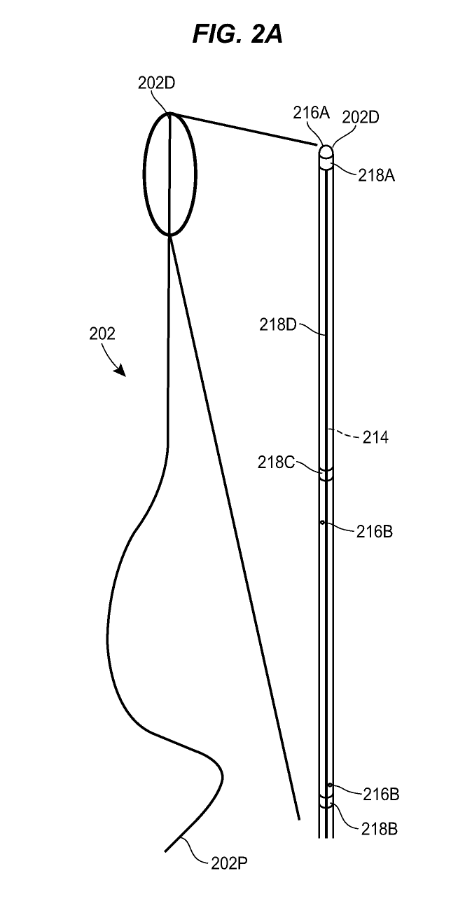

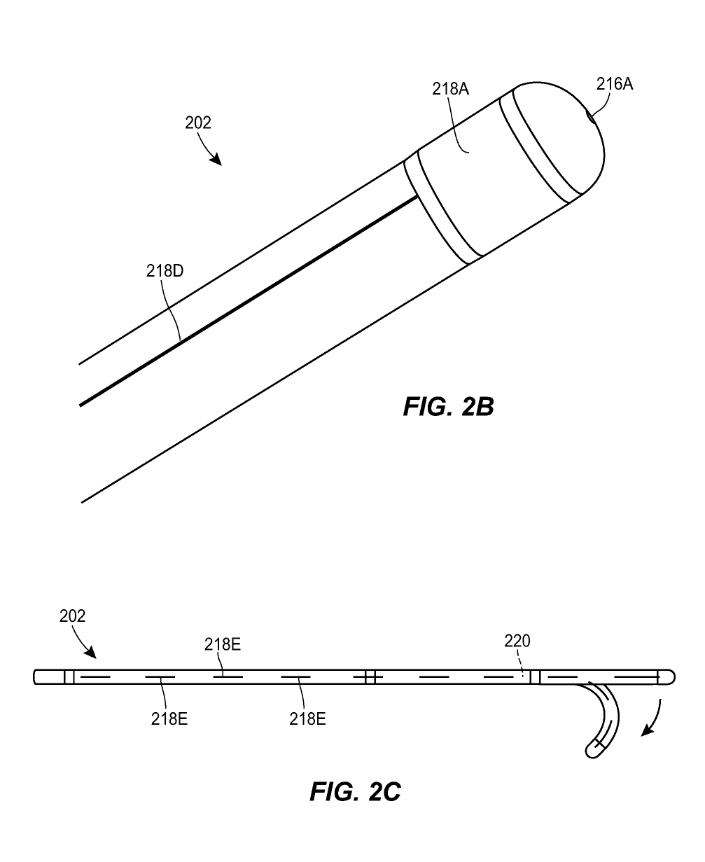

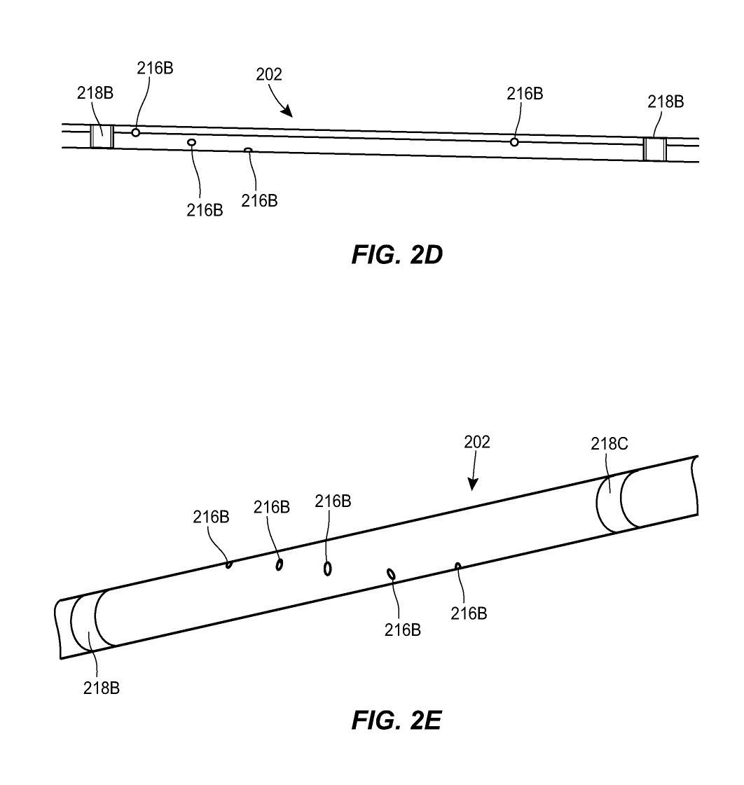

FIGS. 2A-2E illustrate an exemplary catheter 202 that can be used in the system 100. As shown, the catheter 202 can include an elongate body that extends between proximal and distal ends 202p, 202d. The interior of the catheter 202 can define one or more fluid lumens 214 through which fluid can be conveyed through the catheter 202.

The catheter 202 can include one or more fluid ports 216 through which fluid can be communicated between the interior of the catheter and the exterior of the catheter. The catheter 202 can include a distal-facing fluid port 216A formed in terminal distal end surface of the catheter. The catheter 202 can include one or more side facing fluid ports 2166, which can be arranged in any of a variety of patterns and at any of a variety of positions along the length of the catheter. As shown, the catheter 202 can include side fluid ports 2168 arranged in a helical pattern to provide 360 degree staggered outlets. In an exemplary embodiment, the staggered side outlet ports 2168 can extend along a length of the catheter 202 of approximately 2-3 inches and can be spaced approximately 1 inch from the distal end of the catheter.

The catheter 202 can include one or more markers 218. The markers 218 can be visible under CT, MRI, or other imaging techniques. The markers 218 can be observed within captured images of the patient to determine the state, position, and/or orientation of the catheter 202. The markers 218 can be formed from radiopaque materials, metallic materials, or other materials that are visible in patient images. The catheter 202 can include a first marker 218A adjacent to the distal fluid port 216A. For example, the catheter 202 can include a thin marker band 218A disposed just proximal to the distal fluid port 216A. The catheter 202 can include second and third markers 218B, 218C that mark the proximal and distal ends, respectively, of a section of the catheter in which the side fluid ports 2168 are formed. The catheter 202 can include a line marker 218D that runs longitudinally along the catheter. The line marker 218D can be observed to determine whether the catheter 202 is twisted and to what degree the catheter is twisted. The catheter 202 can include length markers 218E spaced in increments along the length of the catheter, e.g., every millimeter, every inch, and/or various fractions thereof. The markers can be embedded in the catheter, inserted into the catheter, formed on an exterior surface of the catheter, attached to the catheter, or otherwise associated with the catheter.

The catheter 202 can be steerable. For example, the catheter 202 can include steering wires or other structures for guiding the distal tip of the catheter within a patient. As shown in FIG. 2C, a curved, bent, or otherwise configured obturator 220 can be inserted through a lumen of the catheter 202 to deflect the distal end of the catheter and thereby steer the catheter.

In some embodiments, the catheter can be a long-term implantable lumbar catheter. The catheter can allow for broad biodistribution in the intrathecal space and can be easily connected to an implantable port. The catheter can be directly attached to the port, or can be connected to the port via an intermediate length of tubing or tubing set. The catheter can be an intrathecal catheter. The catheter can include a single lumen or a plurality of lumens. The catheter can be an intrathecal catheter that can be threaded from lumbar to cervical. The fluid ports of the catheter can be in several configurations including cervical, thoracic, and/or lumbar locations, or as needed for drug distribution.

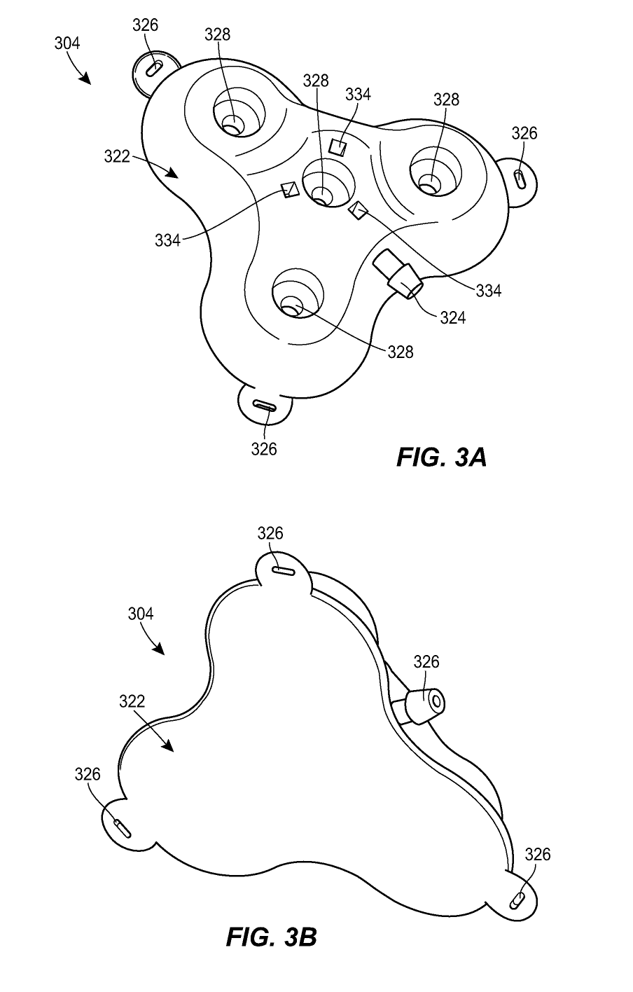

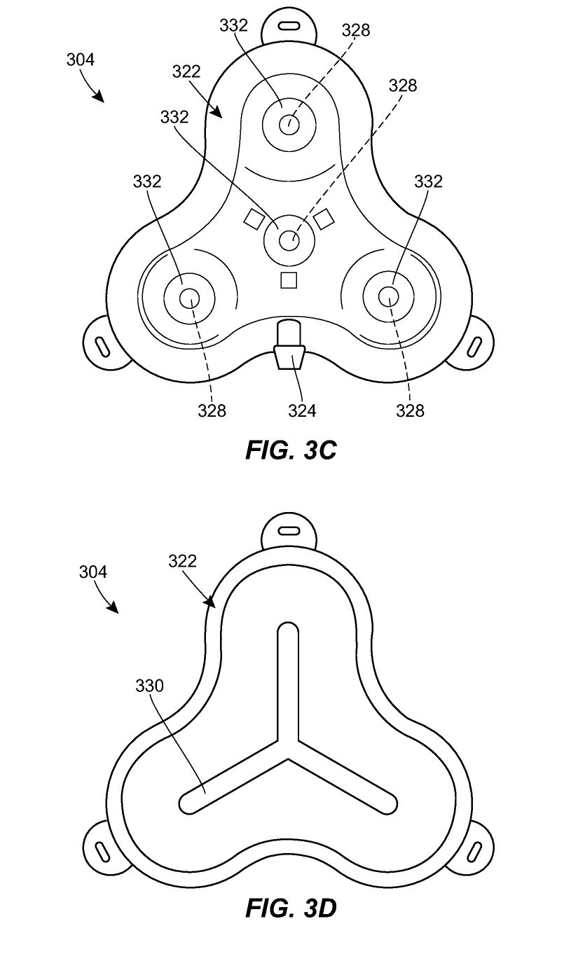

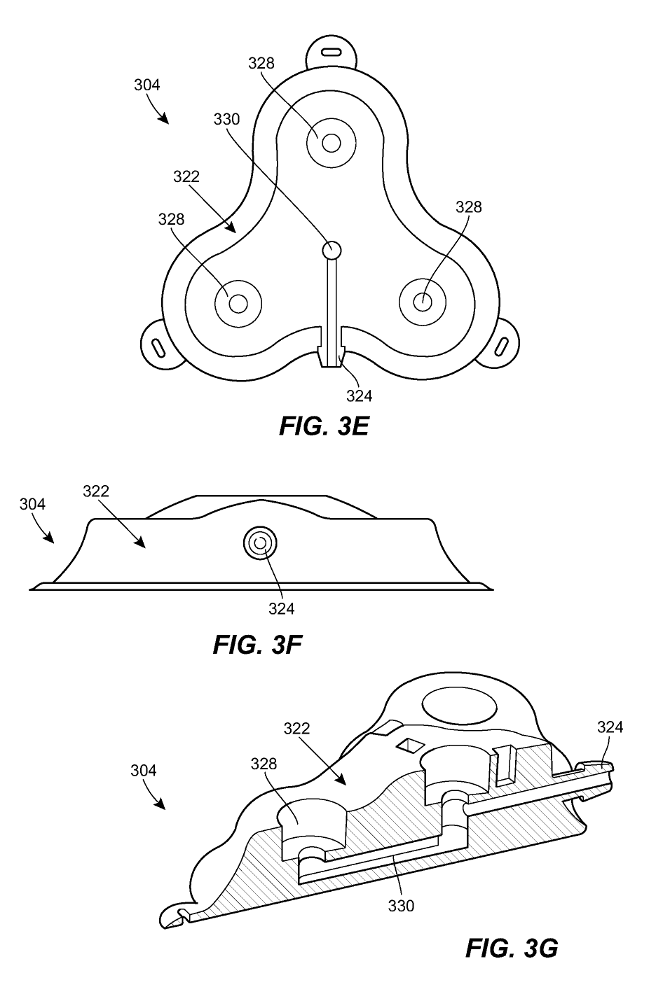

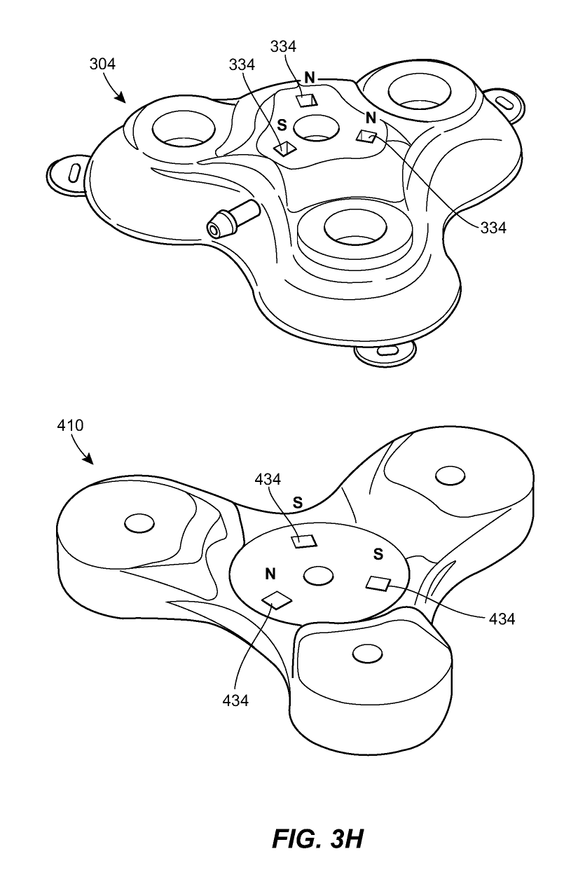

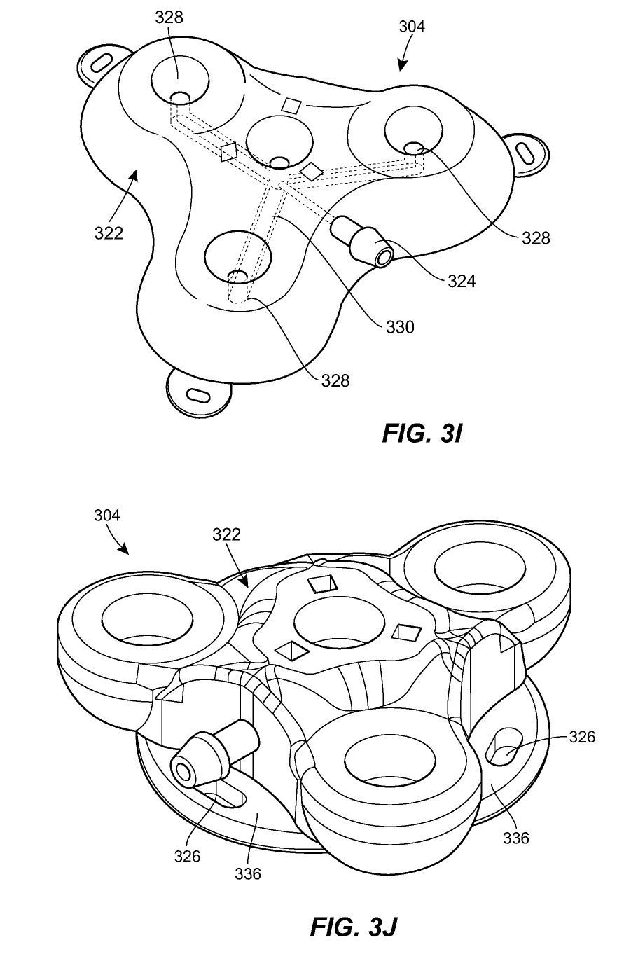

FIGS. 3A-3I illustrate an exemplary port 304 that can be used in the system 100. The port 304 can include a housing 322 that includes a central portion and one or more prongs that extend radially outward from the central portion. The housing 322 can include a coupling 324 for attachment to the catheter or to an intermediate tube or tubing set. While a male barbed fitting is shown, it will be appreciated that any of a variety of other couplings can be used instead or in addition. The catheter can connect directly to the coupling 324, or a short extension tube can be used to connect the catheter to the coupling 324. The extension tube can act as a strain relief for the coupling 324 to reduce any risk of the catheter becoming disconnected from the port.

A lower portion of the housing 322 can be configured to rest on tissue of the patient to support the port 304. The lower portion can include a needle guard to prevent over-insertion of a needle through the port 304. For example, the lower portion can include a layer of material that is resistant to needle penetration, e.g., formed from a metal or rigid polymer. The lower portion can include one or more openings or eyelets 326 for receiving sutures or other fasteners for securing the port 304 to the patient.

An upper portion of the housing 322 can include one or more fluid ports or injection sites 328. Each prong of the housing 322 can include a respective fluid port 328. The central portion of the housing 322 can include a fluid port 328. The fluid ports 328 can be in fluid communication with the catheter coupling 324. For example, as shown in the sectional views of FIGS. 3D, 3E, and 3G and the transparent view of FIG. 3I, the housing 322 can include a network of internal lumens 330 through which fluid can pass between the plurality of fluid ports 328 and the catheter coupling 324. Each fluid port 328 can include a septum 332. The septum 332 can be penetrable by a needle inserted therethrough to allow fluid communication between the needle and the inner lumen 330 of the housing 322. The septum 332 can be configured to form a seal around the inserted needle, and/or to reseal on itself after the needle is removed to prevent leakage of fluid. The septum 332 can be formed from any of a variety of materials, such as silicone, elastomers, and the like. The septum 332 can be a cylindrical plug of material inserted into a corresponding cylindrical recess disposed above the respective fluid port 328.

The upper portion of the housing 322 can include one or more alignment features 334 to facilitate alignment between the port 304 and a connector 410. For example, the housing 322 can include a plurality of magnetic elements 334. As shown in FIG. 3H, the magnetic elements 334 can be arranged to mate with counterpart elements 434 of the connector 410. The magnetic elements 334, 434 can be configured such that the connector 410 can only be mated to the port 304 in a single orientation. For example, as shown in FIG. 3H, one of the magnets 334 in the port 304 can be oriented with an opposite polarity from the others and the magnets 434 of the connector 410 can have the same but opposite orientation. In other words, the port 304 can be configured with the north pole of the magnet 334 facing up in two of the slots and the south pole facing up in the third slot. The connector 410 can be configured with the opposite arrangement, with two slots having the south pole facing out of the connector and the north pole in the third slot facing out. This can allow the connector 410 to only attach to the port 304 in one orientation. If the orientation is off, the connector 410 will not be able to make a tight seal to the port 304 because of the polarity of the magnets 334, 434. The upper portion of the housing 322 can have a sloped surface such that the central portion of the housing is raised as compared to the prongs. The upper portion of the housing 322 can slope upwards towards the center.

As shown in FIG. 3J, the housing 322 can include flanges 336 that span between adjacent prongs. The flanges 336 can include one or more openings or eyelets 326 for receiving sutures or other fasteners for securing the port 304 to the patient.

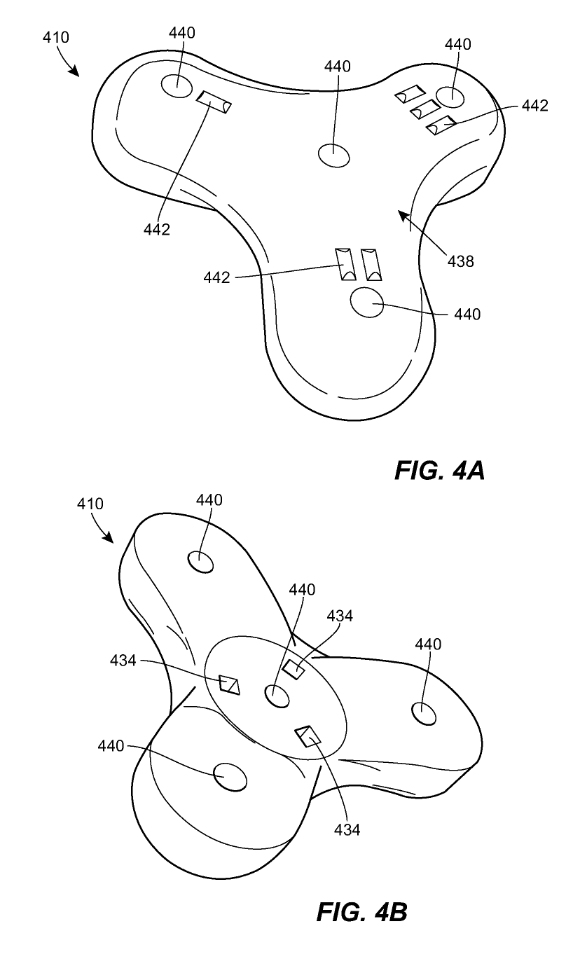

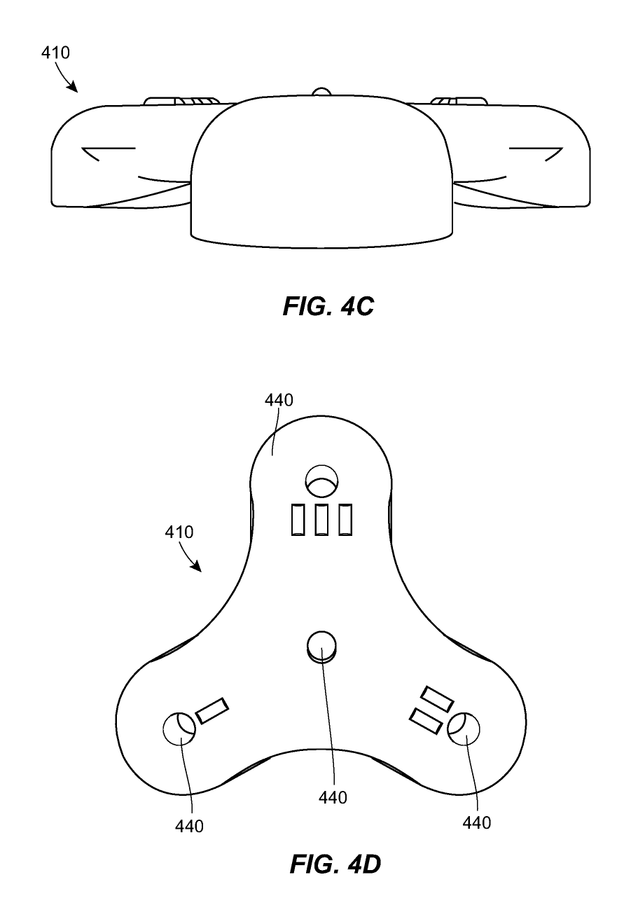

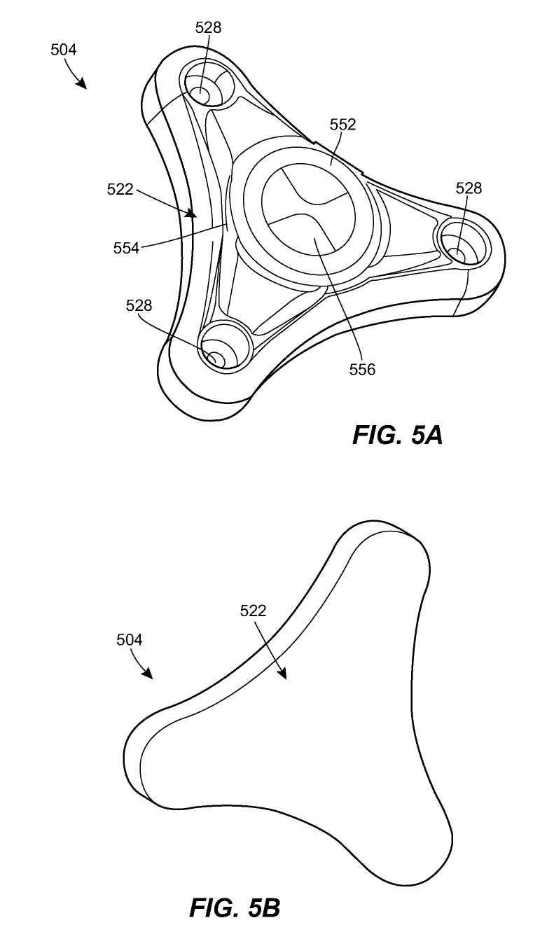

FIGS. 4A-4D illustrate an exemplary connector 410 that can be used in the system 100. The connector 410 can be positioned over a port implanted beneath the surface of a patient's skin, for example, the port 304. The connector 410 can include a housing 438 that matches the shape of the port. For example, the housing 438 can include a central portion and one or more prongs that extend radially outward from the central portion. The connector 410 can include one or more holes 440 extending therethrough from the upper surface of the connector to the lower surface. When the connector 410 is mated to a port, each hole 440 can be aligned with a respective fluid port of the implanted port. The connector 410 can include markings 442 to allow each of the holes 440 to be easily distinguished from one another. This can allow a user to document which ports have been used for previous doses, to ensure an equal or approximately equal use of the plurality of ports, etc. The illustrated markings 442 include a series of tick marks identifying the holes 440 as hole one, hole two, and hole three. It will be appreciated that any of a variety of other markings can be used instead or in addition, such as different color coded marks, different Arabic or Roman numerals, different letters, and/or different geometric shapes.

The lower portion of the connector 410 can include one or more alignment features 434 to facilitate alignment between the connector and an implanted port. For example, the connector 410 can include a plurality of magnetic elements 434. The magnetic elements 434 can be arranged to mate with counterpart elements of the port. As described above, the magnetic elements 434 can be configured such that the connector 410 can only be mated to the port in a single orientation. The lower portion of the connector 410 can have a sloped surface such that the central portion of the connector is depressed as compared to the prongs. The lower portion of the connector 410 can slope upwards towards the center. When mated to the port over a patient's skin surface, at least a portion of the port can be received within the depressed center portion of the lower surface of the connector 410. While magnetic and geometric alignment features are described above, it will be appreciated that any of a variety of other alignment features can be used instead or in addition. For example, tactile alignment features or shape alignment features can be used.

The connector can be configured with a limited number of slots 440 that are open and capable of being injected through. The connector can be configured to selectively open and close one or more of the slots 440, e.g., in accordance with a dosing protocol. Opening and closing of the slots 440 can be electronically controlled, mechanically controlled, etc. In some embodiments, a plurality of different connectors can be provided to the clinician, each having a different arrangement of opened vs. closed slots 440. For example, each connector can be configured such that only one slot 440 is open and capable of being injected through. These features can be beneficial when the dosing protocol is known, as it can allow different connectors to be provided to the clinician in accordance with the dosing protocol. In an exemplary arrangement, in the first dose only slot 1 of the connector will be open, for dose 2 only slot 2 will be open, and so on. The connectors can be provided as a set, e.g., in which a clinician is given two, three, four, or more connectors and throws each connector away or sets it aside after it is used. A “refill” order can be placed to replace used connectors.

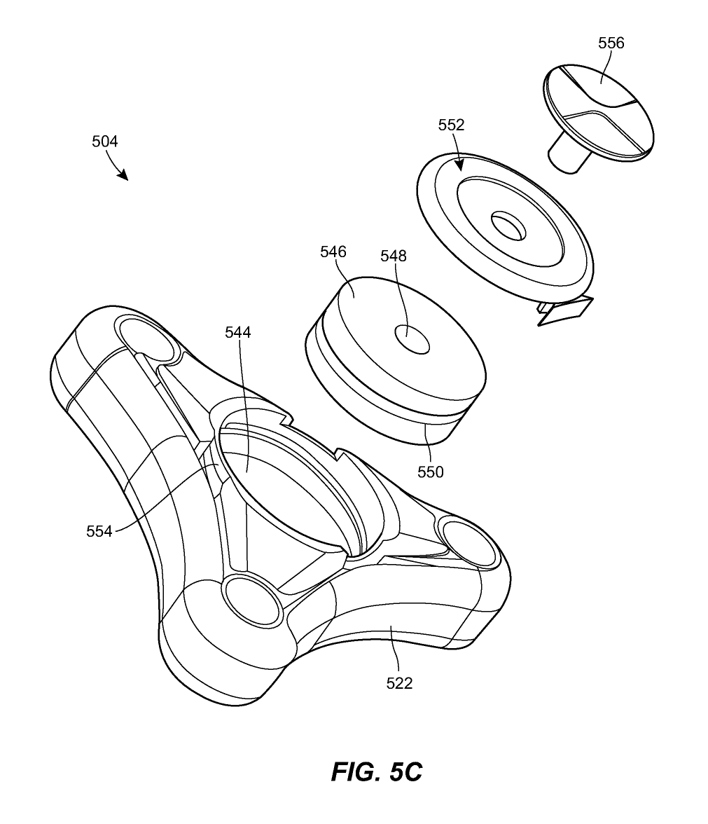

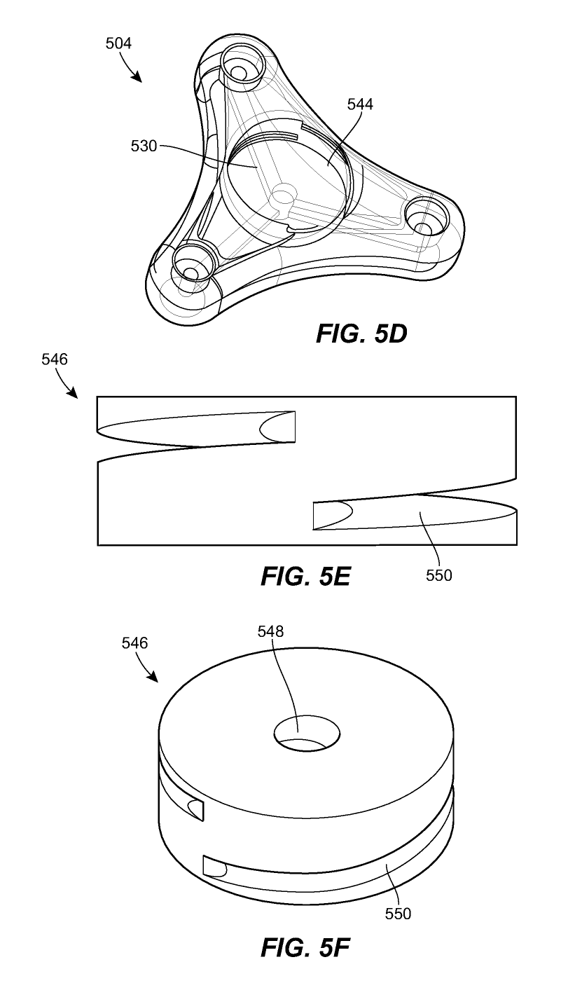

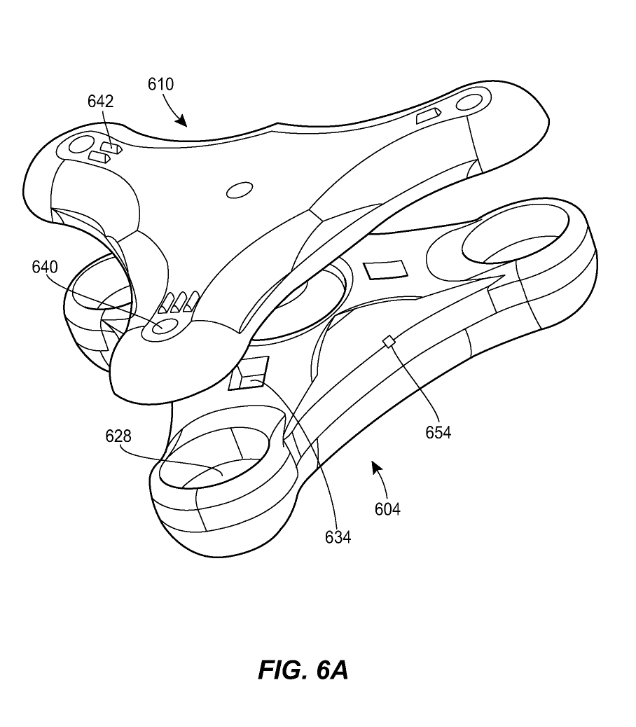

FIGS. 5A-5F illustrate another exemplary port 504 that can be used in the system 100. The port 504 can include any of the features of the other ports described herein, e.g., the port 304. For example, the port 504 can include a housing 522 with a central portion and one or more prongs, each of the prongs having a respective fluid port 528. The housing 522 can include a network of internal fluid lumens 530 that connect each fluid port 528 to a central cavity 544 of the housing. The housing 522 can include a spool 546 rotatably mounted within the cavity 544. A catheter, or a tubing set coupled thereto, can be coiled around the spool 546. The catheter can be in fluid communication with the cavity 544. In use, the spool 546 can be rotated relative to the housing 522 to extend the deployed length of the catheter or to retract the deployed length of the catheter, depending on the direction of rotation. The spool 546 can include a generally cylindrical body with a central opening 548 formed therein. A helical groove 550 can be formed in an outer sidewall of the spool 546. The helical groove 550 can extend all the way to the central opening 548 of the spool 546, or can extend to a lesser depth.

The spool 546 can be retained within the cavity 544 by a cap 552. An opening 554 can be formed at the interface between the cap 552 and the housing 522 through which the deployed section of the spooled catheter can extend out of the housing. The port 504 can include an actuator for rotating the spool 546 relative to the housing 522. For example, the port 504 can include a turnkey 556 as shown. The key 556 can be rotatably mounted in a recess formed in the upper surface of the cap 552. The key 556 can include a shaft that extends distally through the cap 552 and into a central opening 548 of the spool 546. The spool 546 can be rotated to change the deployed length of the catheter by applying a rotational force to the key 556. The force can be applied through manual user manipulation, through an external magnetic field, through an embedded electric motor, or otherwise. The spool 546 can be configured to rotate automatically when tension is applied to the catheter, e.g., due to patient growth or movement.

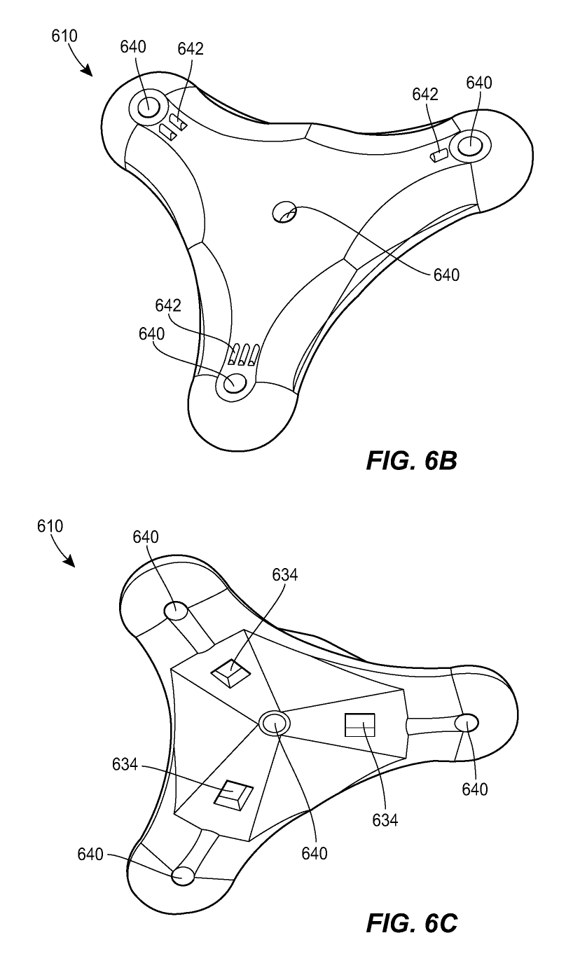

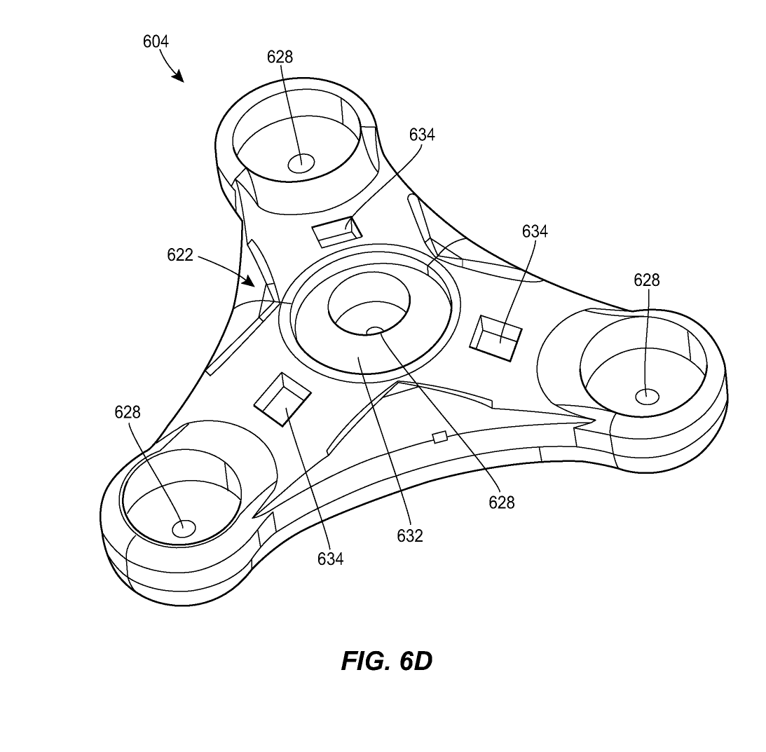

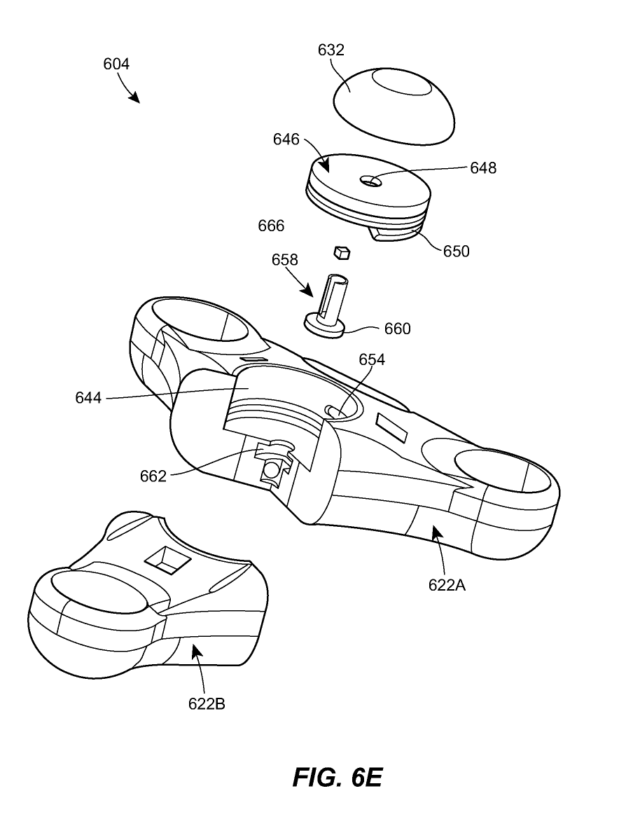

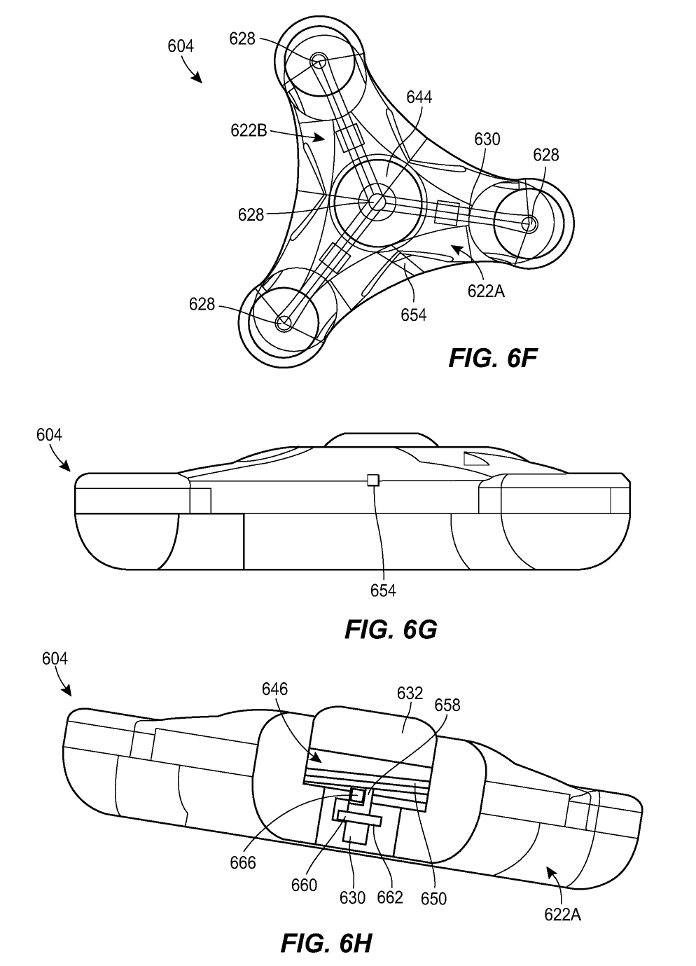

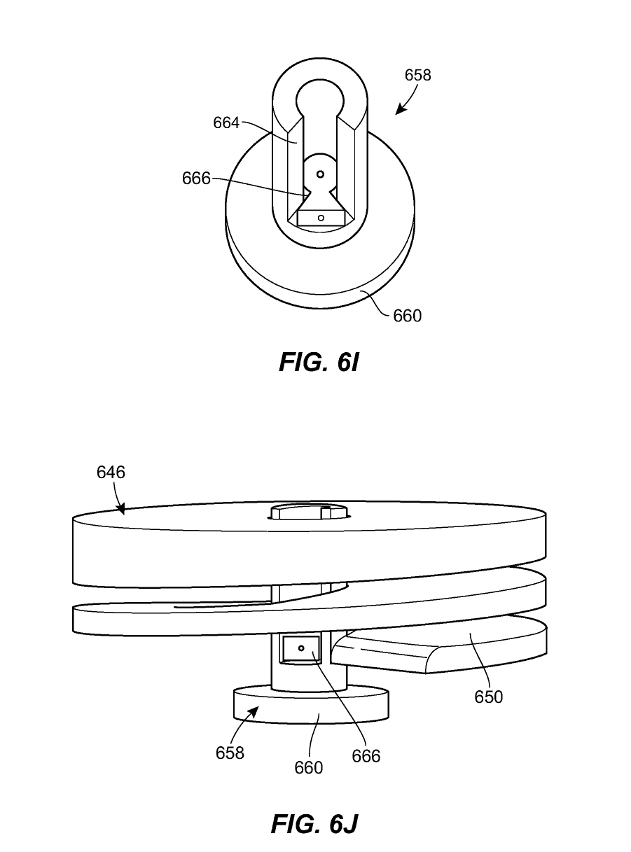

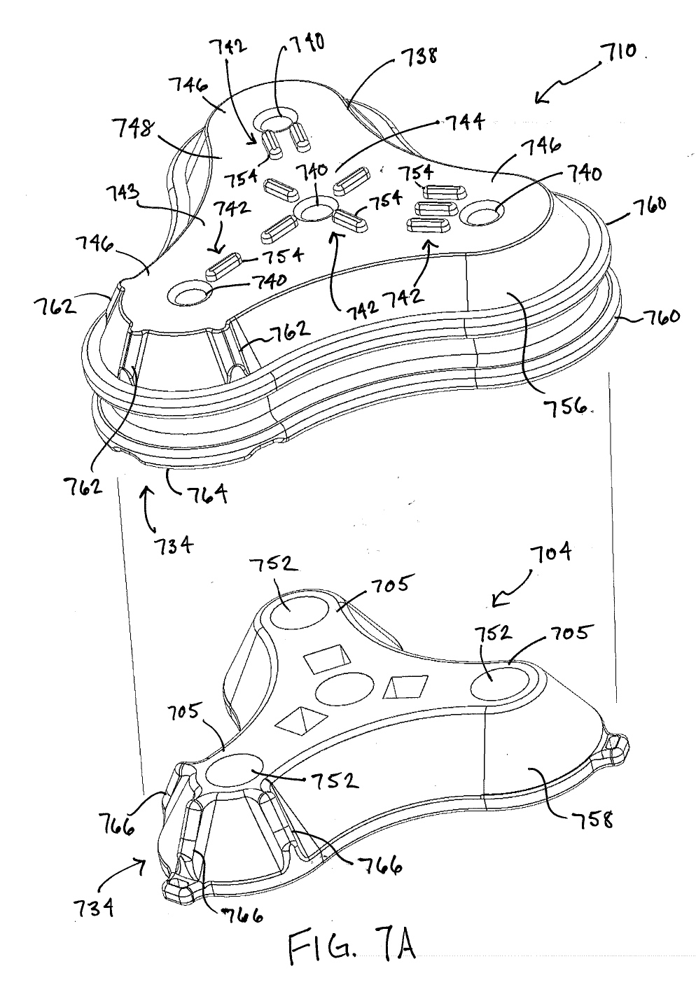

FIG. 6A illustrates another exemplary port 604 and a counterpart connector 610 that can be used in the system 100. The connector 610 can include any of the features of the other connectors described herein, e.g., the connector 410. The connector 610 is shown in greater detail in FIGS. 6B-6C. The connector 610 can include holes 640, markings 642, and alignment features 634, e.g., of the type described above. The port 604 is shown in greater detail in FIGS. 6D-6J. The port 604 can include any of the features of the other ports described herein, e.g., the ports 304, 504. For example, the port 604 can include a housing 622 with a central portion and one or more prongs, each of the prongs having a respective fluid port 628. The housing 622 can include a central fluid port 628. The housing 622 can include a network of internal fluid lumens 630 that connect each fluid port 628 to a central cavity 644 of the housing. The housing 622 can include a spool 646 rotatably mounted within the cavity 644. A catheter, or a tubing set coupled thereto, can be coiled around the spool 646. The catheter can be in fluid communication with the cavity 644, as discussed further below. In use, the spool 646 can be rotated relative to the housing 622 to extend the deployed length of the catheter or to retract the deployed length of the catheter, depending on the direction of rotation. The spool 646 can include a generally cylindrical body with a central opening 648 formed therein. A helical groove 650 can be formed in an outer sidewall of the spool 646. The helical groove 650 can extend all the way to the central opening 648 of the spool 646, or can extend to a lesser depth. An opening 654 can be formed in the housing 622 through which the deployed section of the spooled catheter can extend out of the housing.

The spool 646 can be mounted and/or retained within the cavity 644 by a clutch or shaft 658. The shaft 658 can extend through the central opening 648 of the spool 646 and can include a distal flange or shoulder 660 seated within a groove 662 formed in the housing 622. The shaft 658 can be free to rotate about its central axis relative to the housing 622. The shaft 658 can include a longitudinal slot 664 formed in the outer sidewall thereof. A key 666 can be coupled to the shaft 658 by inserting a cylindrical portion of the key into the central opening of the shaft and positioning a barb protrusion of the key within the longitudinal slot 664 of the shaft. The cylindrical portion of the key 666 and the barb portion of the key can each include a hole formed therethrough to allow fluid flow through the key. A septum 632 can be seated in the cavity 644, above the upper surface of the spool 646. A needle inserted through the septum 632 can be placed in fluid communication with the cavity 644 via a central cannulation of the shaft 658. The proximal end of the catheter, or a tubing extension coupled thereto, can be connected to the key 666 and can be in fluid communication with the cavity 644 via the hole formed in the key. As the deployed length of the catheter is increased, the catheter can unwind from the spool 646, causing the key 666 to travel upwards along the length of the longitudinal slot 664 formed in the shaft 658.

The housing 622 can be an assembly of two or more housing components. For example, as shown, the housing 622 can include a first component 622A that includes two prongs and a second component 622B that includes a single prong. The shaft 658, key 666, spool 646, and/or septum 632 can be assembled to the first component 622A, e.g., by introducing the flange 660 of the shaft 658 laterally into the groove 662 in the housing. The second component 622B can then be secured to the first component 622A to retain the shaft 658, key 666, spool 646, and/or septum 632 therein. The second component 622B can be attached to the first component 622A in various ways, such as a snap-fit connection, sonic welding, an adhesive, or the like.

The spool 646 can be rotated to change the deployed length of the catheter by applying a rotational force to the spool. The force can be applied through manual user manipulation, through an external magnetic field, through an embedded electric motor, or otherwise. The spool 646 can be configured to rotate automatically when tension is applied to the catheter, e.g., due to patient growth or movement.

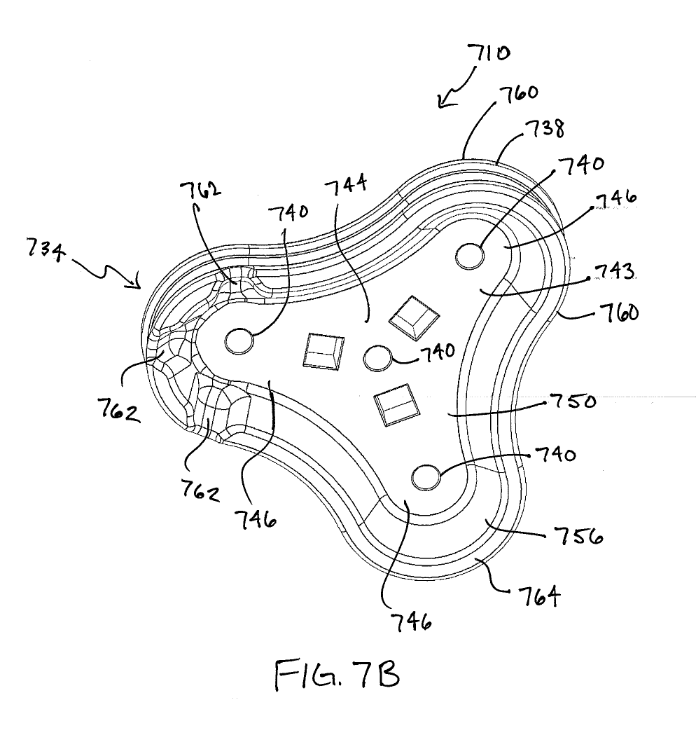

FIGS. 7A and 7B illustrate another exemplary connector 710 that can be used in the system 100. The connector 710 can be positioned over a port implanted beneath the surface of a patient's skin, for example, a port 704 having one or more features of the ports described herein. The connector 710 can include a housing 738 that matches the shape of the port 704. In the illustrated form, a top wall 743 of the housing 738 includes a central portion 744 and one or more prongs 746 that extend radially outward from the central portion 744 that are configured to align with and be complementary to the shape of prongs 705 of the port 704. For example, the housing 738 can include three prongs 746 as shown. Further, the connector 710 can include one or more holes 740 extending therethrough from an upper surface 748 of the connector 710 to a lower surface 750. When the connector 710 is mated to a port, each hole 740 can be aligned with a respective fluid port 752 of the implanted port 704. As with the above forms, the connector 710 can include markings 742 to allow each of the holes 740 to be easily distinguished from one another. This can allow a user to document which fluid ports 752 have been used for previous doses, to ensure an equal or approximately equal use of the plurality of fluid ports 752, etc. The illustrated markings 742 include a series of tick marks 754 identifying the holes 740 as hole one, hole two, hole three, and hole four. It will be appreciated that any of a variety of other markings can be used instead or in addition, such as different color coded marks, different Arabic or Roman numerals, different letters, and/or different geometric shapes.

The connector 710 can also include one or more alignment features 734 to facilitate alignment between the connector 710 and an implanted port 704. In one example, the housing 738 can include a skirt 756 that extends downwardly from the top wall 743. As shown, the skirt 756 can be complementary to a sidewall 758 of the port 704 so that the connector 710 at least partially nests thereover. This advantageously aids a user in locating the port 704 through a patient's tissue. In the illustrated form, the skirt 756 and sidewall 758 are angled outwardly providing easier engagement and alignment of the connector 710 with the port 704. If desired, the skirt 756 can include one or more outwardly protruding flanges 760 to provide a gripping aid to a user trying to locate the port 704 and nest the connector 710 over the port 704.

In a further form, the connector 710 can include a feature to ensure that the housing 738 can be repeatedly and reliably nested over the port 704 in a particular orientation. For example, the skirt 756 can include one or more grooves 762 that extend upwardly from a bottom edge 764 of the skirt 756 towards the top wall 743. The port 704 can include one or more outwardly protruding ribs 766 that extend upwardly along the sidewall 758 thereof. The grooves 762 and ribs 766 are configured to align with one another, such that as the connector 710 is nested over the port 704 over the tissue of the patient, the ribs 766 slide at least partially into the grooves 762. This configuration indicates to a user that the correct prongs of the connector 710 and port 704 are aligned. Although an outer surface of one prong is shown, it will be appreciated that other mating features and locations can be used instead or in addition. The connector 410 can also include a plurality of magnetic elements as described above. The magnetic elements can be arranged to mate with counterpart elements of the port 704 and, as described above, can be configured such that the connector 710 can only be mated to the port 704 in a single orientation.

In any of the ports described herein, the catheter can be coiled around the spool, a length of tubing coupled to the proximal end of the catheter can be coiled around the spool, or both the catheter and a length of tubing coupled thereto can be coiled around the spool. In some embodiments, the spool can have 10-15 cm of tubing and/or catheter wrapped therearound and available to be deployed from the port to extend the effective length of the catheter.

An exemplary method of using the systems described herein can include implanting a catheter within a patient. For example, the catheter can be introduced into a lumbar region of the patient and positioned such that one or more fluid ports of the catheter are disposed in the intrathecal space of the patient. The catheter can be coupled to a port, for example by attaching the catheter to a coupling of the port (in the case of the port 304) or by winding the catheter around a spool of the port (in the case of the ports 504, 604). The port can be implanted beneath the patient's skin. The port can be secured to the patient via sutures or other anchors. Once the port and catheter are in place, any skin incision can be closed using known techniques. Proper positioning and condition of the catheter can be assessed using markers of the catheter and patient imaging. The connector can be selectively coupled to the port, over the patient's skin, when it is desired to infuse or withdraw fluid through the catheter. The connector can be aligned with the port using the alignment features described herein. A needle or syringe can be guided through a hole of the connector, through the patient's skin, and into a fluid port of the implanted port. The needle or syringe can be actuated to infuse fluid into the port and/or catheter, or to withdraw fluid from the port and/or catheter. This process can be repeated, through the same hole or through others, as many times as needed or desired to achieve a specified treatment, dosing protocol, or the like. The connector can be decoupled from the port and removed when injections have been completed, between injections, or at any other desired time.

The systems and methods herein can be used to treat any of a variety of conditions or diseases, including Parkinson's, Friedreich's Ataxia, Canavan's disease, ALS, Congenital Seizures, Drevets Syndrome, pain, SMA, Tauopathies, Huntington's, Brain/Spine/CNS tumors, inflammation, Hunters, Alzheimer's, hydrocephalus (therapeutic cure for hydrocephalus), Sanfillippa A, B, Epilepsy, Epilepsy pre-visualase, PCNSL, PPMS, Acute disseminated encephalomyelitis, Rx of motor fluctuations in advanced Parkinson's patients, Acute repetitive seizures, Status epilepticus, ERT, and/or Neoplastic meningitis. The systems disclosed herein, and/or the component parts thereof, can be designed for therapy specific applications and treatments, particularly but not limited to spinal muscular atrophy, Huntington's disease, ALS, Parkinson's Disease, Alzheimer's Disease, other neurogenerative conditions, and conditions listed above.

The devices described herein are suitable for administering any fluid composition, such as a pharmaceutical composition comprising one or more therapeutic agents, to a subject. Indeed, the device of the disclosure optionally comprises one or more dosages of a therapeutic agent, such as a therapeutic agent suitable for treating (in whole or in part) a disorder, infection, or injury of the central nervous system or spine. Disorders associated with aspects of the central nervous system or spine include, but are not limited to, spinal muscular atrophy, survival motor neuron deficiency, ankylosing spondylitis, spinal tumors, bipolar disorder, encephalitis, depression, epilepsy, Dravet Syndrome, meningitis, multiple sclerosis, myeopathy, Angelman's Syndrome, CNS lymphoma, Leptomeningeal cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN), or neurodegenerative disorders, e.g., Tau protein-related disorders including Alzheimer's disease, Huntington's disease, alpha-synuclei-related disorders including Parkinson's disease, amyotrophic lateral sclerosis (ALS) including superoxide dismutase 1-related ALS, progressive spranuclear palsy, frontotemporal dementia, and Tourette's syndrome. Infections of the CNS include, but are not limited to, viral meningitis, fungal meningitis, epidural infection, viral encephalitis, and neurosyphilis.

Any therapeutic agent may be used in the context of the disclosure. Exemplary therapeutic agents include, e.g., nucleic acids, protein therapeutics, cell therapies, and small molecule therapeutics. Examples of protein therapeutics include antibody-based therapeutics, such as antibodies, antibody fragments, or antibody-like protein products that include binding regions of antibodies (e.g., scFv, diabodies, antibody mimetics, and the like). The antibody-based therapeutic may target, e.g., amyloid plaques, tau proteins, cancer antigens, or abnormal alpha-synuclein. Examples of protein therapeutics also include, but are not limited to, hormones, enzymes (e.g., lysosomal enzymes, such as alpha-L-iduronidase, N-acetylgalactosamine-4-sulfatase, or beta-glucuronidase), growth factors (e.g., fibroblast growth factor (FGF) or neurotrophins or neurotrophic factors, such as glial cell-derived neurotrophic factor (GDNF), brain-derived neurotrophic factor (BDNF), ciliary neurotrophic factor (CNTF), or nerve growth factor (NGF)), blood factors, bone morphogenetic proteins, interferons, interleukins, and thrombolytics. Examples of cell-based therapies include, but are not limited to, stem cell therapeutics and immune cells (including modified immune cells, such as CAR T cells). Suitable small molecule therapeutics include, but are not limited to, analgesics, ion channel blockers, anti-convulsive agents, antibiotics or antiviral agents, anti-inflammatories, anticoagulants, chemotherapeutic, anti-depressants, anti-anxiety agents, steroids, and the like. In various aspects, the therapeutic agent is baclofen, morphine, bupivacaine hydrochloride, clonidine hydrochloride, gabapentin, idursulfase, cytarabine, methotrexate, a corticosteroid, edavarone-conjugate, conotoxin, abomorphine, prednisolone hemisuccinate sodium, carbidopa/levodopa, tetrabenazine, benzodiazepines, such as diazepam and midazolam, alphaxalone or other derivative, cyclophosphamide, idursulfase (Elaprase®), iduronidase (Aldurazyme®), topotecan, buslfan, opmaveloxolone, epicatechin, methylprednisolone, frataxin replacement, reservatrol, nicontinamide, AT-010 (RNA that induces splicing modulation in the mature amyloid precursor protein mRNA), Cerebril™, an anti-Aβ antibody, elenbecestat, a corticosteroid, or nusinersen (Spinraza®), or combinations thereof.

In various aspects, the therapeutic agent is a nucleic acid, including DNA or RNA, which may be single stranded or double stranded and which may be modified or unmodified. Suitable nucleic acid-based therapeutic agents include, but are not limited to, antisense oligonucleotides, ribozymes, miRNA, siRNA, and shRNA. Optionally, the nucleic acid targets a gene selected from the group consisting of APP, MAPT, SOD1, BACE1, CASP3, TGM2, TARDBP, ADRB1, CAMK2A, CBLN1, CDK5R1, GABRA1, MAPK10, NOS1, NPTX2, NRGN, NTS, PDCD2, PDE4D, PENK, SYT1, TTR, FUS, LRDD, CYBA, ATF3, CASP2, HRK, C1QBP, BNIP3, MAPK8, MAPK14, Rac1, GSK3B, P2RX7, TRPM2, PARG, CD38, STEAP4, BMP2, GJA1, TYROBP, CTGF, ANXA2, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, SESN2, SOX9, ASPP1, CTSD, CAPNS1, FAS, FASLG, CAPN1, FADD, CASP1, CASP9, p75NTR, PARK2, HTT (with expanded repeats), NogoA, MAG, OMGP, NgR1, PDE4, BCAN, NCAN, PTPRZ1, TNC, NRP1, NRP2, PLXNA1, PLXNA2, PLXNB1, PLXNC1, TROY, LRRC1, ROCK1, LimK1, LimK2, CFL1, KCNC4, KCNE3, NAT8L, FKBP1A, FKBP4, LRRK2, DYRK1A, AKAP13, UBE2K, WDR33, MYCBP2, SEPHS1, HMGB1, HMGB2, TRPM7, BECN1, THEM4, SLC4A7, MMP9, SLC11A2, ATXN3, ATXN1, ATXN7, PRNP, EFNB3, EPHA4, EFNA5, EPHA7 and EFNB2, such that gene expression or function is modified.

In some embodiments, the therapeutic agent is an oligonucleotide comprising at least one modified nucleotide, optionally a modified nucleotide that reduces binding to cerebral spinal fluid (CSF) proteins. In various embodiments, the modified nucleotide includes a substituent at the 2′-position, such as a 2′-O-2-methoxyethyl (“2′-MOE”) group, as shown below, wherein X is O or S.

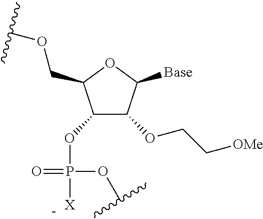

Oligonucleotides comprising a 2′-MOE modification can distribute rapidly in central nervous system tissues. Oligonucleotides comprising such modifications exhibit extended half-lives in CSF and central nervous system tissues, which can result in less frequent dose administration.

In some cases, the modified nucleotide can include a 2′,4′-constrained group, such as a constrained 2′-O-ethyl (“cEt”) group. In various cases, the cEt group can have S-stereochemistry (“S-cEt”), as shown below, wherein X is O or S.

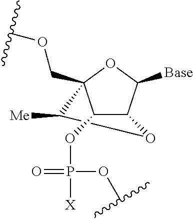

Nucleic acids modified with a constrained ethyl group, such as S-cEt, can exhibit enhanced thermal stability, good potency, and a good therapeutic profile.

Optionally, the nucleic acid encodes a beneficial protein that, e.g., replaces an absent or defective protein, or encodes a cytotoxic protein that achieves a therapeutic effect, such as cancer cell death. Any of the protein-based therapeutics described herein may be delivered to a subject via delivery of a nucleic acid encoding the protein under conditions which allow expression in vivo. For example, in various embodiments, the nucleic acid encodes a neurotrophic factor such as, but not limited to, nerve growth factor (NGF), brain-derived neurotrophic factor (BDNF), neurotrophin-3 (NT-3), neurotrophin-4/5 (NT-4/5), neurotrophin-6 (NT-6), ciliary neurotrophic factor (CNTF), glial cell line-derived neurotrophic factor (GDNF), the fibroblast growth factor family (e.g., FGF's 1-15), leukemia inhibitory factor (LIF), certain members of the insulin-like growth factor family (e.g., IGF-1), a neurturin, persephin, a bone morphogenic protein (BMPs), an immunophilin, a member of the transforming growth factor (TGF) family of growth factors, a neuregulin, epidermal growth factor (EGF), platelet-derived growth factor (PDGF), vascular endothelial growth factor family (e.g. VEGF 165), follistatin, or Hifl, or combinations thereof.

In various aspects, the nucleic acid is present in a viral vector. Any viral vector appropriate for delivering a therapeutic agent to a human subject may be used. Examples of viral vectors include, e.g., herpes simplex virus (HSV) vectors, adenovirus (Ad) vectors, parvoviral-based vectors (e.g., adeno-associated viral vectors), chimeric Ad-AAV vectors, and retroviral vectors (including lentiviral vectors, HIV vectors). Any of these gene transfer vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

In some embodiments, the viral vector is an AAV vector. AAV vectors used for administration of a therapeutic nucleic acid typically have approximately 96% of the parental genome deleted, such that only the terminal repeats (ITRs), which contain recognition signals for DNA replication and packaging, remain. Delivering the AAV rep protein enables integration of the AAV vector comprising AAV ITRs into a specific region of genome, if desired. AAV vectors are useful for delivering payload to the central nervous system due, at least in part, to their safety profile, long-term gene expression, and ability to infect both dividing and quiescent cells, including neurons. Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11. AAV vectors may be engineered to alter the virus's native tropism or improve infection by modifying the viral capsid or packaging the genome of one serotype into the capsid of a different serotype. AAV vectors have been used to deliver a number of transgenes to treat a variety of diseases, including ASP to treat Canavan disease; CLN2 to treat Late infantile neuronal ceroid lipofuscinosis; SGSH to treat mucopolysaccharidosis IIIA; NAGLU to treat mucopolysaccharidosis IIIB; ARSA to treat metachromatic leukodystrophy; GAD, AADC, NTN, GDNF, AADC to treat Parkinson's; and NGF to treat Alzheimer's. See, e.g., Hocquemiller et al., Hum Gene Ther., 27(7), 478-496 (2016), hereby incorporated by reference. The genomic sequences of AAV, as well as the sequences of the ITRs, Rep proteins, and capsid subunits are known in the art. See, e.g., International Patent Publications Nos. WO 00/28061, WO 99/61601, WO 98/11244; as well as U.S. Pat. No. 6,156,303, Srivistava et al. (1983) J Virol. 45:555; Chiorini et al (1998) J Virol. 71:6823; Xiao et al (1999) J Virol. 73:3994; Shade et al (1986) J Virol. 58:921; and Gao et al (2002) Proc. Nat. Acad. Sci. USA 99:11854.

In various embodiments, the device is used to deliver one or more gene editing agents to a subject, such as the clustered regularly interspaced short palindromic repeats (CRISPR) associated protein (Cas) system. CRISPR-Cas and similar gene targeting systems are in the art with reagents and protocols readily available. See, e.g., Mali et al., Science, 339(6121), 823-826 (2013); and Hsu et al., Cell, 157.6: 1262-1278 (2014). Exemplary genome editing protocols are described in Doudna and Mali, “CRISPR-Cas: A Laboratory Manual” (2016) (CSHL Press, ISBN: 978-1-621821-30-4) and Ran et al., Nature Protocols 8(11): 2281-2308 (2013). The CRISPR/Cas system comprises a CRIPSR/Cas nuclease (typically Cas9) and guide RNA (or crRNA-tracrRNA) comprising a short nucleotide targeting sequence that directs the nuclease to a genome location of interest. The guide RNA(s) and coding sequence for the Cas nuclease, optionally packaged into viral vectors, can be delivered to the CSF via the device of the disclosure. The CRISPR/Cas system is further described in, e.g., U.S. Patent Publication Nos. 2018/0223311.

In various aspects, the disclosure provides a method of treating Huntington's disease, Spinal Muscular Atrophy (SMA), survival motor neuron (SMN) deficiency, amyotrophic lateral sclerosis (ALS) (including superoxide dismutase 1 (SOD1)-related ALS), Angelman's syndrome, Dravet syndrome, Alzheimer's disease and other tau protein-related disorders, progressive supranuclear palsy (PSP), frontotemporal dementia (FTD), alpha-synuclei-related disorders including Parkinson's Disease, central nervous system (CNS) lymphoma, leptomeningeal cancer, Friedreich's Ataxia, hereditary cerebral hemorrhage with amyloidosis-Dutch type (HCHWA-D), cerebral amyloid angiopathy (CAA), amyloid congophilic angiopathy (ACA), or secondary malignant neoplasms (SMN). The method comprises implanting a fluid delivery system in the patient such that a catheter of the fluid delivery system is disposed within the patient's intrathecal space. The method further comprises releasing a therapeutic agent (such as any one or more of the therapeutic agents described above) via the catheter into the intrathecal space, such that the disorder is treated.

Additional details on drug delivery systems and methods can be found in U.S. Pat. No. 9,682,193; U.S. application Ser. No. 15/662,416, filed on Jul. 28, 2017; U.S. application Ser. No. 15/849,705, filed on Dec. 21, 2017; and U.S. application Ser. No. 16/192,500, filed on Nov. 15, 2018, each of which is hereby incorporated herein by reference in its entirety.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A fluid delivery system, comprising:
an implantable port having a housing with one or more fluid openings therein; and
a connector configured to be selectively mated to the port over a skin surface of a patient, the connector having one or more openings configured to align with the one or more fluid openings of the port.

2. The system of claim 1, wherein the implantable port further comprises a spool rotatably mounted in the housing, the spool adapted to have portions of a catheter wound therearound.

3. The system of claim 2, wherein the spool comprises a cylindrical body having a helical groove formed in an outer surface thereof.

4. The system of claim 2, wherein the spool is mounted to a shaft having a longitudinal slot with a key slidably disposed therein.

5. The system of claim 4, further comprising a catheter that extends from the port and is in fluid communication with the one or more fluid openings.

6. The system of claim 5, wherein the catheter includes a distal-facing fluid opening and a plurality of side-facing fluid openings, the side-facing fluid openings being arranged in a helical pattern.

7. The system of claim 5, wherein the catheter includes a longitudinal line marker configured to indicate a twist of the catheter.

8. The system of claim 5, wherein the catheter includes markers that indicate proximal and distal ends of a region of the catheter in which side-facing fluid ports are formed.

9. The system of claim 5, wherein the catheter includes a plurality of markers disposed along length increments.

10. The system of claim 5, wherein at least one of the catheter or the port is designed for therapy specific applications and treatments to a disorder selected from the group consisting of Huntington's disease, Spinal Muscular Atrophy (SMA), survival motor neuron (SMN) deficiency, amyotrophic lateral sclerosis (ALS), Angelman's Syndrome, Dravet Syndrome, Alzheimer's disease, progressive supranuclear palsy (PSP), frontotemporal dementia (FTD), Parkinson's Disease, central nervous system (CNS) lymphoma, Leptomeningeal Cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN).

11. The system of claim 4, wherein the key includes an opening extending therethough, and the catheter is connected to the key and in fluid communication with the opening.

12. The system of claim 4, wherein the key is configured to shift along the longitudinal slot of the shaft as the spool rotates to unwind a catheter would therearound.

13. The system of claim 4, wherein housing includes a groove; and the shaft includes a radial flange disposed within the groove to retain the shaft to the housing.

14. The system of claim 1, wherein the one or more fluid openings of the housing each include a respective septum.

15. The system of claim 1, wherein the one or more fluid openings of the housing are in fluid communication with one another via an inner lumen of the housing.

16. The system of claim 1, wherein the housing and the connector include counterpart alignment features.

17. The system of claim 16, wherein the alignment features are at least one of magnetic, tactile, and shape alignment.

18. The system of claim 1, wherein the housing includes a top surface and a side surface; and the connector comprises a top wall having a shape complementary to the top surface of the housing and a skirt depending downwardly from the top wall, such that with the connector selectively mated to the port over a skin surface of a patient, the skirt extends along portions of the side surface of the housing.

19. The system of claim 18, wherein the housing further comprises one or more ribs protruding outwardly from the side surface thereof; and the skirt of the connector further comprises one or more grooves each adapted to at least partially receive one of the one or more ribs therein with the connector mated to the port over a skin surface of a patient.

20. The system of claim 1, wherein the housing includes a male barbed catheter fitting adapted to receive a catheter thereover to fluidly couple the catheter to the housing.

21. The system of claim 1, wherein the connector comprises a housing have a shape complementary to a shape of the housing of the implantable port.

22. The fluid delivery system of claim 1, further comprising one or more dosages of a nucleic acid, a protein therapeutic, a cell therapy, a small molecule therapeutic, or a combination thereof.

23. The fluid delivery system of claim 22, comprising a nucleic acid selected from the group consisting of an antisense oligonucleotide, a ribozyme, an miRNA, an siRNA, and and shRNA, or a nucleic acid encoding a clustered regularly interspaced short palindromic repeats (CRISPR) associated protein (Cas) system, or a combination thereof.

24. The fluid delivery system of claim 23, wherein the nucleic acid is an antisense oligonucleotide comprising a 2′-O-2-methoxyethyl (“2′-MOE”) group.

25. The fluid delivery system of claim 23, comprising an antisense oligonucleotide, and the antisense oligonucleotide is nusinersen.

26. The fluid delivery system of claim 23, comprising an antisense nucleic acid that targets HTT.

27. The fluid delivery system of claim 22, comprising one or more dosages of a viral vector encoding a therapeutic protein.

28. The fluid delivery system of claim 27, wherein the viral vector is an adeno-associated viral vector or an adenoviral vector.

29. The fluid delivery system of claim 22, wherein the nucleic acid, protein therapeutic, cell therapy, small molecule therapeutic, or combination thereof treats a disorder selected from the group consisting of Huntington's disease, Spinal Muscular Atrophy (SMA), survival motor neuron (SMN) deficiency, amyotrophic lateral sclerosis (ALS), Angelman's Syndrome, Dravet Syndrome, Alzheimer's disease, progressive supranuclear palsy (PSP), frontotemporal dementia (FTD), Parkinson's Disease, central nervous system (CNS) lymphoma, Leptomeningeal Cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN).

30. A fluid delivery system, comprising:
an implantable port having a housing with one or more fluid openings therein; and
a catheter that extends from the port and is in fluid communication with the one or more fluid openings, the catheter including a distal-facing fluid opening and a plurality of side-facing fluid openings, the side-facing fluid openings being arranged in a helical pattern.

31. The system of claim 30, wherein the catheter includes a longitudinal line marker configured to indicate a twist of the catheter.

32. The system of claim 30, wherein the catheter includes markers that indicate proximal and distal ends of a region of the catheter in which the side-facing fluid openings are formed.

33. The system of claim 30, wherein the catheter includes a plurality of markers disposed along length increments.

34. The system of claim 30, wherein the housing includes a male barbed catheter fitting adapted to receive a catheter thereover to fluidly couple the catheter to the housing.

35. The system of claim 30, wherein the one or more fluid openings of the housing each include a respective septum.

36. The system of claim 30, wherein the implantable port further comprises a spool rotatably mounted in the housing, the spool adapted to have portions of the catheter wound therearound.

37. The system of claim 36, wherein the spool comprises a cylindrical body having a helical groove formed in an outer surface thereof.

38. The system of claim 36, wherein the spool is mounted to a shaft having a longitudinal slot with a key slidably disposed therein.

39. The system of claim 30, wherein at least one of the catheter or the port is designed for therapy specific applications and treatments to a disorder selected from the group consisting of Huntington's disease, Spinal Muscular Atrophy (SMA), survival motor neuron (SMN) deficiency, amyotrophic lateral sclerosis (ALS), Angelman's Syndrome, Dravet Syndrome, Alzheimer's disease, progressive supranuclear palsy (PSP), frontotemporal dementia (FTD), Parkinson's Disease, central nervous system (CNS) lymphoma, Leptomeningeal Cancer, Friedreich's Ataxia, hereditary cerebral hemorrhage with amyloidosis-Dutch type (HCHWA-D), cerebral amyloid angiopathy (CAA), amyloid congophilic angiopathy (ACA), and secondary malignant neoplasms (SMN).

40. A fluid delivery system, comprising:
an implantable catheter;
means for establishing fluid communication between the implantable catheter and a needle; and
means for aligning the needle with the means for establishing fluid communication.
